(19) United States
(12) Patent Application Publication
(10) Pub. No.: US 2009/0099638 A1
(43) Pub. Date: Apr. 16, 2009

(54) MOTORIZED DEPLOYMENT SYSTEM

(75) Inventor: David D. Grewe, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc., West Lafayette, IN (US)

(21) Appl. No.: 12/236,744

(22) Filed: Sep. 24, 2008

Related U.S. Application Data
(60) Provisional application No. 60/979,337, filed on Oct. 11, 2007.

(51) Int. Cl. A61F 2/06; A61B 19/00

(52) U.S. Cl. 623/1.11; 128/898

(57) ABSTRACT

A motorized delivery system and method for deploying an endoluminal prosthesis is disclosed. The system comprises a delivery device and an electrical drive system. The prosthesis is disposed between an inner dilator and an elongate sheath. To deploy the prosthesis, the electrical drive system is actuated. One or more gear-pulley arrangements rotate to cause retraction of the sheath in relation to the inner dilator.

### RELATED APPLICATIONS

This application claims the benefit of priority from U.S. Provisional Application No. 60/979,337 filed Oct. 11, 2007, which is incorporated herein by reference.

### FIELD OF THE INVENTION

This invention relates to a medical device and, in particular to a delivery device for a self-expanding prosthesis and a method of delivering and deploying a prosthesis into a body lumen.

### BACKGROUND

Endoluminal prostheses are used for treating damaged or diseased body lumens such as the esophagus, bile duct, and blood vessels. For example, endoluminal prostheses are used for repairing diseased aorta including abdominal aortic aneurysms and thoracic aortic aneurysms.

An endoluminal device or prosthesis may be placed inside the body lumen to provide some or all of the functionality of the original, healthy vessel. Methods of placing a prosthesis inside a body lumen include surgical repair and endovascular repair. Endovascular repair generally involves percutaneous placement of the prosthesis, for example a stent graft, using a catheter delivery device. An incision is made in the patient to provide vascular access, for example, through the femoral artery. A delivery device, including a radially-compressed prosthesis, is inserted through the incision and the prosthesis is delivered to the area to be treated. The prosthesis is released from the delivery catheter and is expanded to engage the body lumen, thereby supporting the lumen and excluding the aneurysm.

A method for deploying an endoluminal prosthesis into the lumen of a patient from a remote location by the use of a catheter delivery device involves radially compressing the endoluminal prosthesis by an outer sheath. To deploy the prosthesis, the operator moves the outer sheath proximally over the prosthesis. The prosthesis expands outwardly upon removal of the sheath. Such a delivery device has been referred to as a “push-pull” system because as the operator pulls the sheath proximally in relation to the prosthesis that is mounted on an inner dilator, the prosthesis is pushed out of the sheath by the inner dilator. Such delivery devices may be advantageous because they can be provided with a relatively small profile, thereby minimizing potential trauma to the patient. A drawback to such delivery devices is that the individual components may be very tightly interconnected, creating high frictional drag and making it difficult to manually retract the sheath from the prosthesis. An exemplary delivery device may require as much as 100 Newtons or approximately 22.5 pounds of force to slide the sheath over the inner dilator and the prosthesis. Such resistance is highly undesirable and can easily tire the operator.

Some delivery devices include an actuation handle that provides a mechanical advantage to the operator. The sheath is retracted by first rotating the handle about an axis of the delivery system and then by sliding the handle proximally. The actuation handle of these delivery devices can be mechanically complicated and still require a fair amount of physical exertion by the operator.

Other delivery devices utilize hydraulic fluid to retract a cover from a prosthesis. The retraction device is disposed within the cover adjacent the prosthesis in an annular space between the cover and the inner dilator. The retraction device is configured to travel within the body lumen. The operator deploys the device remotely by injecting hydraulic fluid through the inner dilator to the retraction device. Such a delivery device is mechanically complex. The position of the retraction device within the cover is inconvenient and may negatively affect the profile of the delivery device. Because the retraction device is completely disposed within the body lumen, the deployment device cannot be deployed in the event of malfunction during the procedure.

Delivery devices, such as those described above may be characterized by a high deployment effort. This is due, in part, to the fact that the sheath frictionally engages the prosthesis and the inner dilator over a relatively large surface area. Where the prosthesis is self-expanding, it will be biased in contact with the sheath, thereby increasing the deployment resistance. Additionally, the hemostatic sealing device must tightly couple the sheath to the inner dilator in order to prevent blood loss during a procedure, increasing the deployment resistance. This accumulation of resistive components can result in a delivery system that is difficult to manually deploy and poses a substantial challenge to designing such push-pull delivery systems.

In view of the drawbacks of current technology, there is a desire for a delivery system that can reduce the deployment effort. Although the inventions described below may be useful for reducing the efforts incurred during deployment of an expandable prosthesis, the claimed inventions may also solve other problems.

### SUMMARY

The invention may include any of the following aspects in various combinations and may also include any other aspect described below in the written description or in the attached drawings.

In a first aspect, an electrical drive system for retracting a sheath from an inner dilator in a prosthesis delivery and deployment system is provided. The drive system comprises a motorized assembly, the motorized assembly being removably coupled to the sheath so that actuation of a motor causes the sheath to slide with respect to the inner dilator.

In a second aspect, a motorized delivery system for delivering and deploying an expandable endoluminal prosthesis is provided. The system comprises an inner dilator having a proximal end and a distal end. An elongate outer sheath is also provided that has a proximal end, a distal end, and an inner lumen defining an inner surface. The distal end of the sheath is slidably disposed over the inner dilator. An electrical drive mechanism is also provided comprising a motorized pulley assembly, the assembly being removably coupled and in mechanical communication with the sheath, whereby actuation of a motor causes the motorized pulley assembly to pull the elongate sheath proximally over the inner dilator.

In a third aspect, a method of deploying an expandable endoluminal prosthesis is provided. The method comprises the steps of providing a prosthesis delivery system comprising an expandable prosthesis, an inner dilator and an elongate sheath. The prosthesis is disposed in a compressed configuration between the inner dilator and the elongate sheath. An electrical drive system is also provided comprising a motorized assembly having a motor, gear assembly, and a pulley assembly, the electrical drive system being removably coupled to a proximal end of the delivery system. The steps include actuating the motor, thereby driving the pulley assembly, and retracting the sheath.

These and various other aspects of the invention can be better understood from the following description with reference to the accompanying figures. The components in the figures are not necessarily to scale, emphasis instead being placed upon illustrating the principles of the invention. Moreover, in the figures, like referenced numerals designate corresponding parts throughout the different views.

### BRIEF DESCRIPTION OF THE DRAWINGS

FIG. 1 is a block diagram of an electrical drive system adapted to be removably coupled to a delivery device;

FIG. 2 is a side elevation view of a delivery device coupled to the electrical drive system;

FIG. 3 is a perspective view of the electrical drive system;

FIG. 4 is a longitudinal cross-sectional view of the delivery device coupled to the electrical drive system; and

FIGS. 5-9 are cross-sectional views of a motorized delivery system deploying an expandable prosthesis in a body lumen.

### DETAILED DESCRIPTION OF THE PREFERRED EMBODIMENTS

Throughout the specification, the terms “distal” and “distally” shall denote a position, direction, or orientation that is generally toward the patient. Accordingly, the terms “proximal” and “proximally” shall denote a position, direction, or orientation that is generally away from the patient.

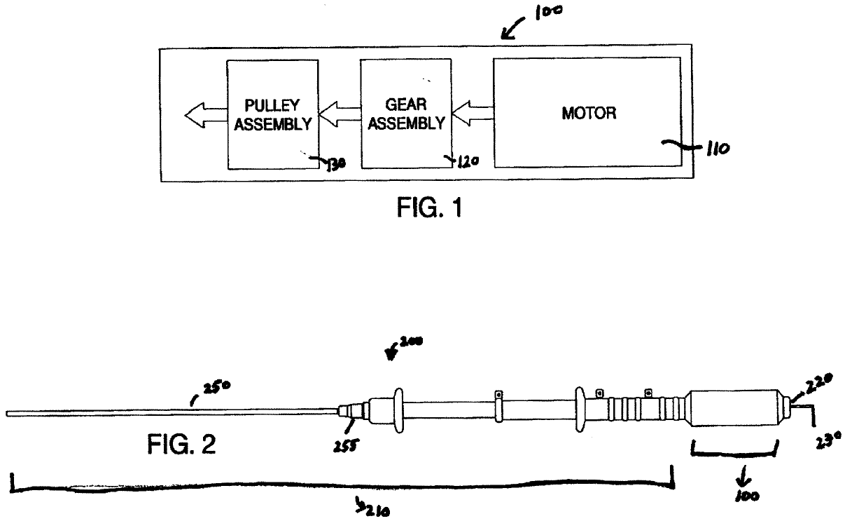

FIG. 1 shows an electrical drive system 100 that may be removably coupled to a delivery device. Actuation of the electrical drive system 100 causes automatic retraction of a delivery sheath thereby causing deployment of an expandable prosthesis into a body lumen. Preferably, the electrical drive system 100 is designed as a modular component that may be removed from the delivery device.

FIG. 1 shows that the electrical drive system 100 comprises a motor 110, a gear assembly or gear box 120, and a pulley assembly 130. The motor 110 is in electrical communication with the gear box 120, and the gear box 120 is in mechanical communication with the pulley assembly 130. The gear box 120 may act to gear down the output of the motor shaft to decrease motor rpm speed and correspondingly increase the torque (i.e., turning power) of the motor 110. In other words, the gear box 120 converts the relatively high rpm of the motor shaft to a relatively low rpm with high torque. The resultant higher torque of the electrical drive system 100 may apply sufficient force to overcome the frictional resistance between the delivery device and the sheath to retract the sheath, which will be explained in greater detail below.

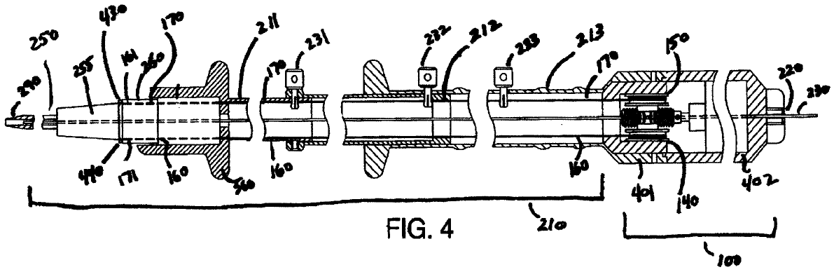

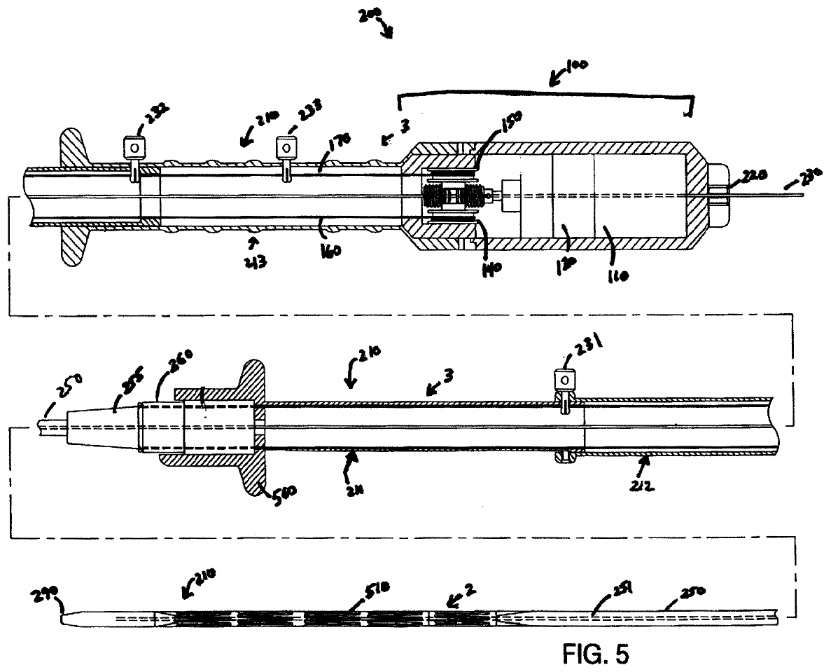

The electrical drive system 100 may be removably coupled to a variety of delivery devices to form a motorized delivery system. One example is shown in FIG. 2. FIG. 2 shows an electrical drive system 100 removably coupled to a delivery device 210 to form a motorized delivery system 200. A sheath 250 is shown slidably disposed over the distal end of the delivery device 210. A sleeve 255 is coupled to the proximal end of the delivery sheath 250. The sleeve 255 is coupled to the pulley assembly 130 by a structure that is detailed in FIGS. 4-9. An expandable prosthesis 510 (FIG. 5) is disposed within the sheath 250. The sheath 250 is slidably disposed over an inner dilator 251 (FIG. 5). A bore 220 extends through the central axis of the system 200 for a tubular housing 230 to be inserted therethrough. The bore 220 may be provided through the electrical drive system 100 that extends through the central axis of the electrical drive system 100 for a tubular housing and a guidewire to extend therethrough. The bore 220 is configured to align with the guidewire lumen of the delivery device 210 to form a continuous passage for a guidewire to extend therethrough. The guidewire may be advanced through the tubular housing 230, which extends the entire longitudinal length of the system 200. Although a bore 220 is shown extending through the drive system 100, the drive system 100 may alternatively be designed without a bore 220.

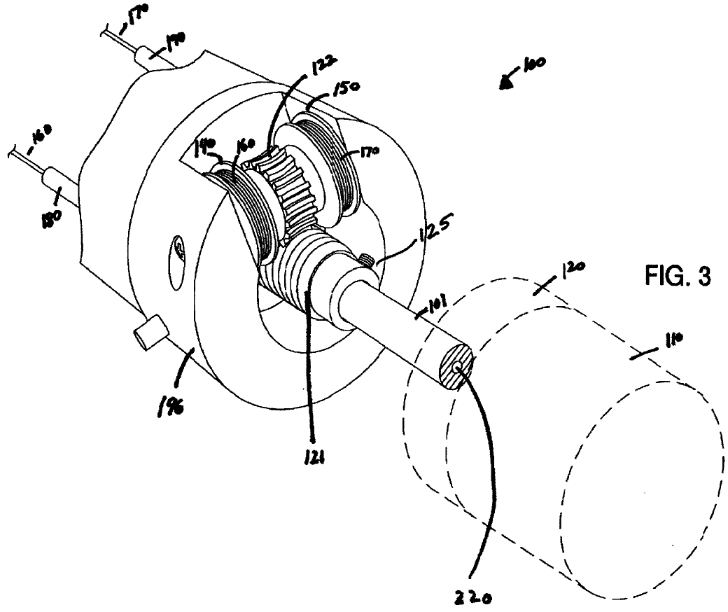

Referring to FIG. 3, the various components of the electrical drive system 100 will now be discussed. In this example, the motor 110 is a 6 V, 2 ampere DC motor that operates at about 6000 rpm. The motor 110 may run on four 1.5 V lithium batteries that are connected in series. It should be appreciated that motors with other operating levels of voltages and amperages may be utilized. The motor 110 has a switch that initiates the deployment of a prosthesis. The example of FIG. 3 shows that the motor 110 has a bore 220 extending along its entire length for the tubular housing 230 and guidewire therewithin to be inserted. The bore 220 may align with a guidewire lumen of the delivery device. Although the motor 110 has been shown with a bore 220, other motors that do not have a bore may be utilized.

FIG. 3 shows that a gear box 120 may be provided to convert the high rpm (i.e, about 6000 rpm) generated by the motor 110 to a relatively lower rpm, higher torque drive system. In particular, the motor shaft may be mechanically coupled to a shaft entering the gear box 120. The gear box 120 gears down the motor shaft such that the shaft 101 (FIG. 3) exiting the gear box 120 has decreased speed (i.e., rpm) but increased turning power (i.e., torque). An oil seal on the shaft 101 may be provided to substantially prevent oil in the gear box from entering into the delivery device 210 (FIG. 2).

Still referring to FIG. 3, a worm 121 engaged to a worm gear 122 is shown. The worm 121 of this example is a quad worm in which a new spiral is formed about every 90°. Generally speaking, the number of cut spirals (i.e., threads) on the worm 121 may be a direct multiplier in terms of the speed that the worm gear turns. A quad worm may rotate the worm gear about four times faster than a typical single cut worm (i.e, 1 cut spiral every 360°) does. Increased rotation of the worm gear 122 may result in faster retraction of the sheath 250. Although the example being discussed utilizes a quad worm, other types of worms may be utilized. For example, a worm having six or eight cut spirals about every 90° may be utilized.

The worm 121, as shown in FIG. 3, may be coaxially coupled over the output shaft 101 of the gear box 120. A set screw 125 may mechanically couple the worm 121 to the output shaft 101. The worm 121 extends along the central axis of the delivery device 210 (FIG. 2). The worm 121 contains a central bore 123 through which a tubular housing 230 may pass. The guidewire (not shown) extends into the housing 230. The worm 121 is driven by the rotation of shaft 101, which is actuated by the gear assembly 120 which is actuated by the motor 110.

A worm gear 122 is shown disposed above the worm 121 and substantially perpendicular to the worm 121 (FIG. 3). The worm gear 122 is driven by rotation of the worm 121. The worm gear 122 is shown as a cylindrical gear with complimentary teeth that mate or mesh with the corresponding threads of the worm 121. The tooth spacing and angle of the teeth of worm gear 122 are substantially identical to the thread spacing and angle of threads of the worm 121 to enable meshing between the two. The mating and movement of the teeth of the worm gear 122 with the spirals of the worm 121 enables incremental retraction of the sheath 250. Such incremental retraction of the sheath 250 (FIG. 5) may provide placement precision of the prosthesis as compared to conventional delivery devices and systems. The drive axis of the worm gear 122 is oriented at about 90° from the drive axis of the worm 121. The drive axes of the worm 121 and worm gear 122 operate on non-intersecting perpendicular axes.

FIG. 3 shows that the worm gear 122 is situated between two pulleys 140 and 150. Generally speaking, pulleys 140 and 150 are linear conversion mechanisms that convert rotational motion to linear motion. The worm gear 122 and pulleys 140, 150 are shown as rotatably secured (e.g., set screwed) to an axle. The worm 121 drives worm gear 122, which causes pulleys 140 and 150 to rotate. Specifically, rotation of the worm gear 122 causes the axle to turn which causes the pulleys 140 and 150 to turn. The axle extends through the worm gear 122 and pulleys 140, 150. Each end of the axle secures into the housing 196 (FIG. 3). Bearings or bushings may be coupled to the ends of the axle for increased stabilization. As compared with conventional delivery devices, the relatively smaller size of the worm 121 and worm gear 122 arrangement may enable the electrical drive system 100 to be compact and portable, thereby allowing the electrical drive system 100 to be readily attached and detached from various delivery devices. Although FIG. 3 shows a worm-worm gear arrangement, other types of gears and configuration of gears (with or without a bore extending therethrough depending on whether an axial guide wire is used in the procedure) are contemplated, such as, for example, spur gears and bevel gears.

Although two pulleys 140 and 150 are shown, the electrical drive system 100 may contain a single pulley or more than two pulleys. The number of pulleys to be used may be dependent upon numerous factors including the force needed to slide the sheath 250 over the inner dilator 251 and the prosthesis 510, as well as the magnitude of a bending moment that the system 200 (FIG. 2) can withstand. Preferably, a minimum of two pulleys may be utilized to minimize the bending moment and distribute the force incurred by valve housing 260 (FIG. 4). As will be explained in greater detail below, the distal ends of the cables 160 and 170 are affixed to a valve housing 260, which is affixed to the sheath 250 (FIG. 4). As the cables 160 and 170 pull on the valve housing 260, the sheath 250 retracts. Utilizing two pulleys may help to counteract the pull incurred on one side of the valve housing 260 with the pull incurred one another side of the valve housing 260, thereby substantially eliminating any bending moment experienced by the system 200. Alternatively, four pulleys and two axles may be utilized in which two pulleys are rotatably disposed about an axle. The ability to design a four pulley system may be limited by the amount of space available in the particular electrical drive system 100. Notwithstanding the above design considerations, a single pulley and cable arrangement may be utilized. Because of the relatively large bending moment incurred by the valve housing 260 with a single pulley, it may be preferable to utilize a relatively more rigid delivery shaft.

Referring to FIG. 3, each of the pulleys 140 and 150 are shown with respective cables 160 and 170 wound therearound. Cable 160 is shown to wind around pulley 140. Cable 170 is shown to wind around pulley 150. The cables 160 and 170 extend along the longitudinal axis of the delivery device 210, as shown in FIGS. 4-9. The distal ends 161 and 171 (FIG. 4) of each of the cables 160 and 170 may affix to the portion of the delivery device 210 (e.g., sleeve 255) that is coupled to the delivery sheath 250. In this example, the distal end of the cables 160 and 170 are affixed to the valve housing 260. When pulleys 140 and 150 rotate, they drive their respective cables 160 and 170. In particular, rotation of the pulleys 140 and 150 causes the pulleys 140 and 150 to exert a tensile force on the cables 160 and 170. The tensile force on the cables 160 and 170 causes them to pull on the valve housing 260, which is affixed to sheath 250, thereby retracting the sheath 250 and ultimately deploying the prosthesis, as will be explained in greater detail below. Cables 160 and 170 may be formed from stainless steel. However, other materials are contemplated.

The cables 160 and 170 may extend away from the electrical drive system 100 along the delivery device 210 (FIGS. 3-9). In this example, the distal end 161 of cable 160 and the distal end 171 of cable 170 (FIG. 4) may be affixed to the wall of the valve housing 260 of the delivery device 210 by copper ferrules 430 and 440 (FIG. 4). The valve housing 260 as shown may be affixed to the sleeve 255, which may be coupled to the delivery sheath 250. When the pulleys exert a tensile force on the cables 160 and 170, the cables 160 and 170 may pull on the sheath 250, thereby exposing the expandable prosthesis. Each of the cables 160 and 170 are shown housed in a tubing 180 and 190 (FIG. 3) to facilitate advancement of the cables 160 and 170 along the longitudinal axis of the delivery device 210 during assembling and loading of the delivery system 210. After the cables 160 and 170 and their respective tubing 180 and 190 have advanced through the segments 211, 212, and 213 of the delivery device 210, the cables 160 and 170 may exit their respective tubing 180 and 190 and enter into the valve housing 260 (FIGS. 4-9). Other means of advancement and attachment of the distal ends 161, 171 of the cables 160 and 170 to the delivery device 210 are contemplated.

The electrical drive system 100 may be modular in design. Referring to FIG. 4, the motor 110 and gear box 120 are contained in a housing 402 and the pulleys 140, 150 with their respective wound cables 160 and 170 are contained in another housing 401, as shown in FIG. 4. The housing 401 may contain two pins that each slidably interlock into a respective bayonet locking device of the motor housing 402. Other connections of the gear housing 401 to the motor housing 402 are contemplated. The distal end of the housing 401 may be mechanically coupled to engage with the proximal end of the delivery device 210 in any way. The compactness of the modular design of the electrical drive system 100 allows it to be coupled to various delivery devices to create a motorized delivery system.

The delivery device 210 may be a stent graft introducer, as shown in FIGS. 4-9. The delivery device 210 comprises a prosthesis delivery section 2 and an external manipulation section 3 (FIG. 5). Although the Figures will be explained in reference to a stent graft, it should be understood that all of the embodiments may apply to other types of expandable prostheses. The delivery section 2 travels through the body lumen during the procedure and delivers the prosthesis 510 to a desired deployment site. The external manipulation section 3 stays outside of the body during the procedure. The external manipulation section 3 can be manipulated by the operator to position and release or deploy the prosthesis 510 into the body lumen.

The delivery device 210 comprises an inner dilator 251 and an elongate tubular sheath 250. The inner dilator 251 and the sheath 250 may be separate slidably interconnected tubes that are configured to selectively retain and release an expandable prosthesis 510 as shown in FIG. 5. Sheath 250 is slidably disposed over the prosthesis 510. The sheath 250 assists in retaining and compressing the expandable prosthesis 510 therewithin. During loading and assembly of the delivery device 210, the sheath 250 can be advanced over the inner dilator 251 while the prosthesis 510 is held in a compressed state by the radially compressive force of the sheath 250. The sheath 250 and inner dilator 251 extend proximally to the manipulation region 3.

The distal end of the delivery device 210 may have an atraumatic head 290 disposed on the distal end thereof (FIGS. 4-9). The atraumatic head 290 may be distally tapered to provide for atraumatic insertion into the body lumen. A guidewire lumen may extend longitudinally through the inner dilator 251 between the proximal and distal ends. The guidewire extends through the bore 220 of the electrical drive system 100 and into the guidewire lumen of the delivery device 210.

The delivery device 210 also comprises three segments, 211, 212, and 213 (FIGS. 4 and 5). Segment 211 is coaxially slidable into segment 212, and segments 211 and 212 are coaxially slidable into segment 213. The segments 211, 212 and 213 slidably dispose with respect to each other as a result of the sheath 250 retracting, as will be explained below. The longitudinal length of delivery device 210 decreases as the segments 211, 212 and 213 are coaxially slidably disposed within each other.

The first segment 211 has a valve socket 560 (FIGS. 4, 5) and into this receives the valve housing 260. The sheath 250 is affixed to the valve housing 260 and extends distally to the atraumatic head 290 of the inner dilator 251 (FIG. 5). Proximal of the atraumatic head 290 is located the expandable prosthesis 510 (FIG. 5). The valve housing 260 may be retained in the valve socket 560 on the first segment 211 by means of a male Luer connector which locks into valve socket 260 (not shown). When release pin 231 is removed, the first segment 211 can slide within the second segment 212. When release pin 231 is locked into the delivery device 210 as shown in FIG. 5, the release pin 231 prevents slidable movement of the first segment 211 within the second segment 212.

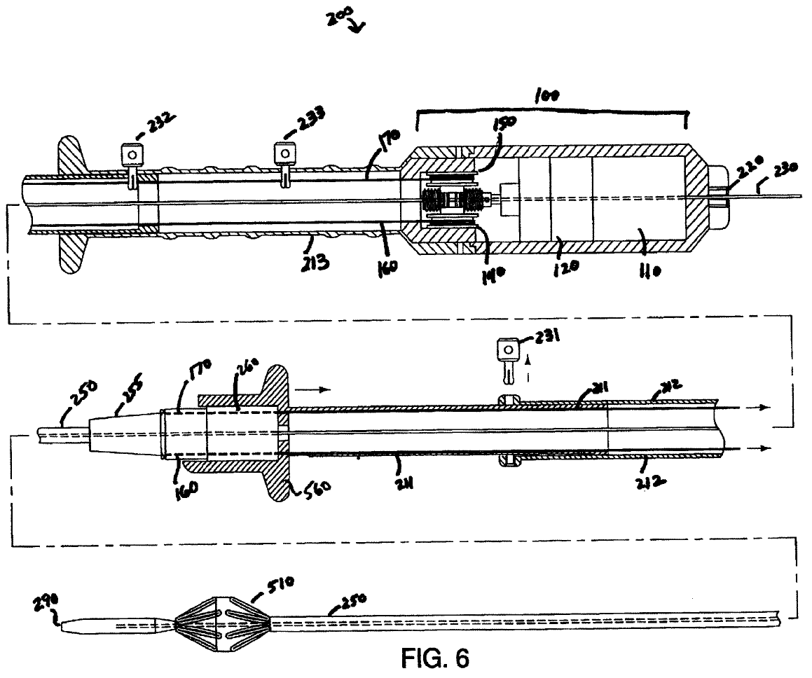

The prosthesis 510 is retained in a radially reduced configuration between the inner dilator 251 and the sheath 250 (FIG. 6). The sheath 250 is slidably disposed over the prosthesis 510 and the inner dilator 251 in a proximal and a distal direction. In particular, the sheath 250 may be slid proximally with respect to the inner dilator 251 and the prosthesis 510 to expose the prosthesis 510 or it may be slid distally over the prosthesis 510 to cover the prosthesis 510.

The prosthesis 510 may comprise a biocompatible graft material. Examples of suitable graft materials include polyesters, such as poly(ethylene terephthalate), polylactide, polyglycolide and copolymers thereof; fluorinated polymers, such as polytetrafluoroethylene (PTFE), expanded PTFE and poly(vinylidene fluoride); polysiloxanes, including polydimethyl siloxane; and polyurethanes, including polyetherurethanes, polyurethane ureas, polyetherurethane ureas, polyurethanes containing carbonate linkages and polyurethanes containing siloxane segments.

The prosthesis 510 may additionally or alternately comprise a stent or a series of stents. Stents may be self-expanding or balloon-expandable. A balloon-expandable stent or stent portion may be combined with a self-expanding stent or stent portion. Self expanding stents can be made of stainless steel, materials with elastic memory properties, such as nitinol, or any other suitable material. A suitable self-expanding stent includes Z-STENTS®, which are available from Cook, Incorporated, Bloomington, Ind. USA. Balloon-expandable stents may be made of stainless steel (typically 316LSS, CoCr, Etc.).

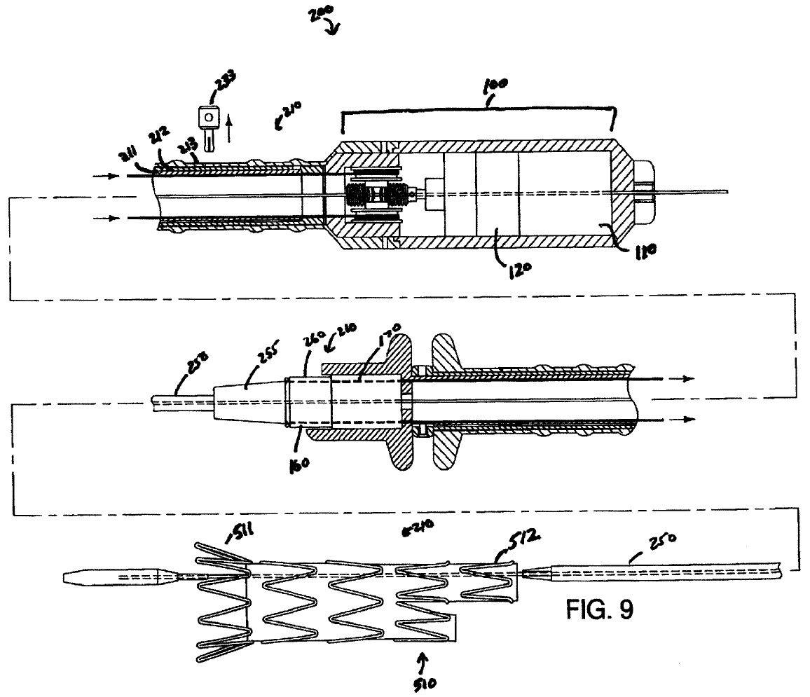

As shown in FIG. 9, the prosthesis 510 can comprise a stent graft having a plurality of self-expanding stents. The self-expanding stents cause the prosthesis 510 to expand during its release from the delivery device 210. The stents may be at least partially covered by a graft material. The prosthesis 510 also may include an exposed self-expanding stent, which is a bare wire stent. The stent may comprise barbs that extend from the stent. When the stent is released, the barbs anchor the end of the prosthesis 510 to the surrounding lumen (not shown). The ends of the prosthesis 510 may be retained by a fastening to which is locked a trigger wire (not shown) which extends to a trigger wire release mechanism (not shown).

The sheath 250 comprises an elongate tubular body having a proximal and distal end and a sheath lumen. The sheath lumen has a generally constant diameter between the proximal and distal ends. The inner dilator 251 is slidably disposed within the sheath lumen. The sheath 250 extends proximally from the delivery section 2 to the user manipulation section 3. The sheath 250 releasably covers the prosthesis 510 in a radially reduced configuration. The atraumatic head 290 and the sheath 250 preferably form a generally smooth transition so as to prevent trauma to the body lumen during insertion. The proximal end of the delivery device 210 is configured to remain outside of the body during the procedure and can be directly manipulated by the operator to deploy the prosthesis 510.

The sheath 250 may be made of any suitable biocompatible material, for example PTFE. The sheath 250 may optionally be provided with a flat wire coil (not shown) to provide the sheath 250 with superior flexibility and kink-resistance.

The delivery device 210 may further comprise two hemostatic sealing devices (not shown) contained in valve housing 260. The hemostatic sealing devices are configured to provide a hemostatic seal between the inner dilator 251 and the sheath 250 to reduce blood loss during a procedure. The hemostatic sealing devices preferably includes three check flow valves, although fewer than three or greater than three check flow valves may be used. A seal ring may also be provided. The seal ring may form a sufficiently tight hemostatic seal around the inner dilator 251. The check flow valves may provide sufficient frictional resistance during deployment. Other hemostatic devices are contemplated and may be utilized in the design of the delivery device 210.

Having described the various components of the motorized delivery system 200, a method of deploying an expandable prosthesis 510 with the motorized delivery system 200 can now be discussed with respect to FIGS. 5-9. A guidewire may initially be advanced through tubular housing 230 (FIG. 2), which longitudinally extends the entire length of the delivery system 200. As the prosthesis 510 in its compressed state squeezes down tightly over the inner dilator 251, advancement of the guidewire therethrough may be difficult. Accordingly, the tubular housing 230, which may be a stainless steel tubing, receives the guidewire and facilitates advancement of the guidewire along the delivery device 210, particularly through the prosthesis 510. The delivery system 200 is then inserted through the body lumen over the guidewire and positioned by radiographic techniques that are generally known in the art. At this stage, the prosthesis 510 is fully retained in the delivery device 210 in a radially-constrained configuration by the sheath 250 as shown in FIG. 5. Since the prosthesis 510 is properly loaded within the delivery device 210, the motorized delivery system 200 can be advanced to the target site.

After advancing the delivery system 200 to the target site, deployment may begin. Prior to actuating the motor 110, the motorized delivery system 200 is in the configuration shown in FIG. 5. The delivery sheath 250 is completely unretracted, thereby fully retaining the prosthesis 510 in its compressed state within the sheath 250. Each of the three 211, 212 and 213 segments of the delivery device 210 is shown locked in their unretracted positions via release pins 231, 232 and 233.

Actuation of the motor 110 (FIG. 3) causes the motor shaft to rotate. The gear box 120 gears down the motor shaft such that the output shaft 101 (FIG. 3) from the gear box 120 has increased torque or turning power. The rotation of the shaft 101 in a particular direction rotates the worm 121 (FIG. 3) in the same direction. Rotation of the worm 121 drives the worm gear 122 (FIG. 3) in the same direction as the worm 121. The worm gear 122 causes pulleys 140 and 150 to rotate. With cables 160 and 170 wound around the pulleys 140 and 150, the pulleys 140 and 150 pull on their respective cables 160 and 170, thereby exerting a tensile force on their respective cables 160 and 170.

Referring to FIG. 6, with the motor 110 actuated, release pin 231 is manually withdrawn (as indicated by the upwards arrow) from the first segment 211, thereby allowing electrical drive system 100 to automatically slidably retract the first segment 211 in the proximal direction (as indicated by the arrow pointing towards the proximal direction) into the second segment 212. In particular, as the pulleys 140 and 150 rotate, they begin to wind their respective cables 160 and 170 around the pulleys 140 and 150. This winding creates a tensile force within each of the cables 160 and 170. The tensile force exerted by the rotating pulleys 140 and 150 on their respective cables 160 and 170 causes the cables 160 and 170 to pull on the valve housing 260, which is connected to the sleeve 255. Because the sleeve 255 is mechanically coupled to the sheath 250, the sheath 250 is pulled a predetermined amount in the proximal direction. The retraction of the sheath 250 has resulted in a portion of the first segment 211 of the delivery device 210 slidably moving into the second segment 212, thereby shortening the overall longitudinal length of the delivery device 210. The electrical drive system 100 provides sufficient force to overcome the frictional resistance between the delivery device 210 and the sheath 250. Partial retraction of the sheath 250 disengages the middle portion of the prosthesis 510 so that it can expand radially, as shown in FIG. 6. Because the ends of the prosthesis 510 still remain affixed to the delivery device 210, the prosthesis 510 can be repositioned for accurate placement within the body lumen.

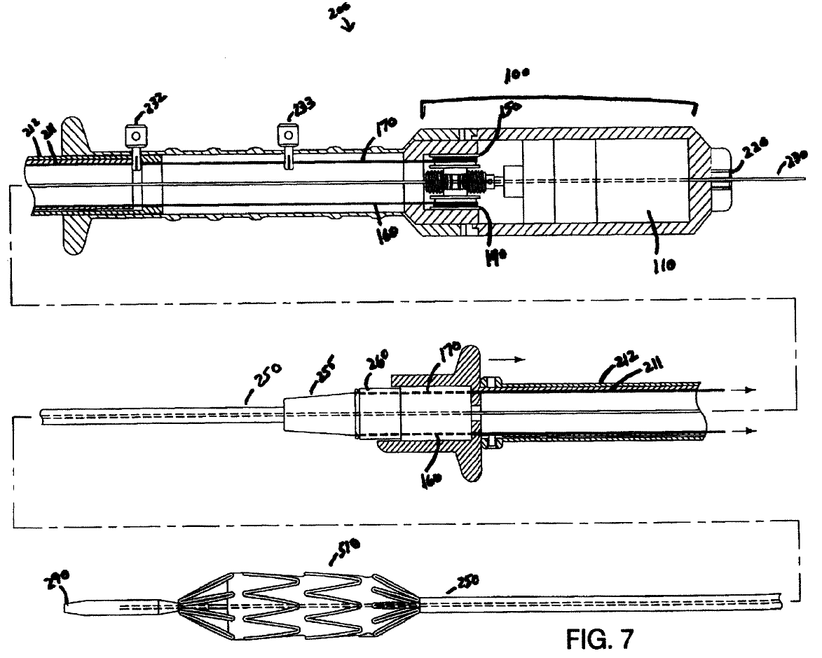

Referring to FIG. 7, with the motor 110 still actuated, the first segment continues to slidably retract in the proximal direction into the second segment (as indicated by the arrows pointing toward the proximal direction). The pulleys 140 and 150 continue to rotate and wind more of their respective cables 160 and 170 around the pulleys 140 and 150. This winding provides tension exerted by the pulleys 140 and 150 on the cables 160 and 170, thereby causing the cables 160 and 170 to pull on the valve housing 260, sleeve 255 and sheath 250 in the proximal direction. FIG. 7 shows that the first segment 211 has been entirely slidably disposed within the second segment 212. At this juncture, the motor 110 may be deactuated so that the partially deployed stent graft may be positioned as desired within the body lumen of the patient. After such positioning, the first segment 211 and the second segment 212 are still engaged with the second release pin 232, which is located at the interface of the first and second segments 211 and 212. A greater portion of the middle portion of the prosthesis 510 has been exposed, so that it can expand radially. Because the ends of the prosthesis 510 still remain affixed to the delivery device 210, the prosthesis 510 can still be repositioned for accurate placement within the body lumen.

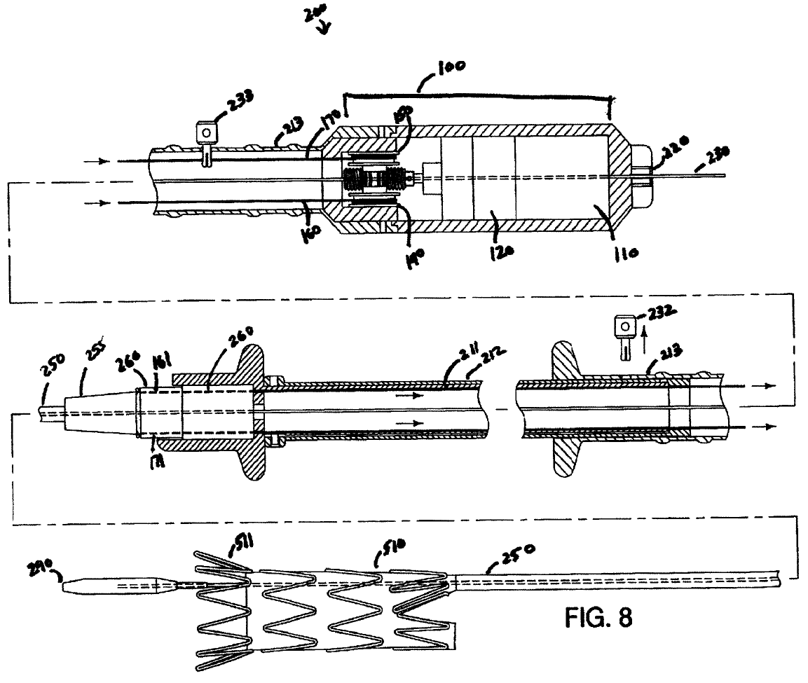

Referring to FIG. 8, with the motor 110 actuated, the second release pin 232 may be manually withdrawn from the interface of the first and second segments 211 and 212, thereby allowing electrical drive system 100 to automatically slidably retract the first and second segments 211 and 212 in the proximal direction (as indicated by the arrow) into the third segment 213. FIG. 8 shows that a portion of the first and second segments 211 and 212 has been slidably coaxially disposed into the third segment 213. The valve housing 260, which contains the distal ends 161, 171 of the cables 160, 170, continues to move in the proximal direction. The continual rotation of the pulleys 140 and 150 continues to wind their respective cables 160 and 170 around the pulleys 140 and 150, thereby causing the cables 160 and 170 to further proximally pull the sheath 250. The electrical drive system 100 continues to provide sufficient force (i.e., torque) to overcome the frictional resistance between the delivery device 210 and the sheath 250 and between the inner dilator 251 and the check flow valves and the seal ring. The sheath 250 has been withdrawn to the extent that the main body of the prosthesis 510 is exposed along with a side leg. At the same time, proximal movement of the first and second segments 211 and 212 has pulled the trigger wires from their retention arrangement (not shown) to free the distal end 511 of the prosthesis 510 from the delivery device 210.

Referring to FIG. 9, the third release pin 233 may be manually withdrawn from the third segment 213 to allow the first and second segments 211 and 212 to fully slide within the third segment 213. Complete deployment of the prosthesis 510 occurs when the proximal end 512 of the prosthesis 510 has been disengaged from the delivery device 210 by removal of proximal trigger wires (not shown) as known in the art. Release of the proximal and distal ends of the prosthesis 510 cause the self-expanding stents of the prosthesis 510 to expand. Hooks on the self-expanding stents grip into the walls of the lumen of the patient to anchor the prosthesis 510 in place. FIG. 9 shows the delivery device 210 in a fully deployed state. The delivery device 210 is in a shortened configuration with the sheath 250 completely retracted from the prosthesis 510, and the prosthesis 510 fully expanded within the body lumen. The motorized delivery system 200 may now be removed from the body lumen along with the electrical drive system 100. The guidewire may remain in place for subsequent procedures.

As can be seen, retraction of sheath 250 occurs incrementally, thereby allowing the physician to continue adjustment of the placement of the prosthesis 510. The motorized delivery system 200 is designed such that the prosthesis 510 may be manually deployed if desired. Although the electrical drive system 100 has been described with respect to delivery device 210, it should be understood that the electrical drive system 100 may also be used with other types of delivery devices.

A varying speed motor may be utilized to further control the rate of retraction of the sheath 250. To this end, the operator may manually adjust the motor setting. The sheath withdrawal rate could range from about 1 mm/sec to about 10 mm/sec. Preferably, the rate is about 5 mm/sec. Design features such as motor speed, the number of teeth on the worm gear 122, the number of spiral cuts on the worm 121, and the diameters of the pulley, worm gear, and worm collectively help to provide greater control of sheath 250 retraction as compared with conventional delivery devices.

Throughout this specification various indications have been given as to preferred and alternative embodiments of the invention. However, it should be understood that the invention is not limited to any one of these. It is therefore intended that the foregoing detailed description be regarded as illustrative rather than limiting, and that it be understood that it is the appended claims, including all equivalents, that are intended to define the spirit and scope of this invention.

1. An electrical drive system for retracting a sheath from an inner dilator in a prosthesis delivery and deployment system, the drive system comprising a motorized assembly, the motorized assembly being removably coupled to the sheath so that actuation of a motor causes the sheath to slide with respect to the inner dilator.

2. The electrical drive system according to claim 1, further comprising a conversion mechanism for converting the motor actuation into sheath retraction.

3. The electrical drive system according to claim 2, wherein the conversion mechanism comprises a pulley and a cable, the cable extending from the pulley to the sheath.

4. The electrical drive system according to claim 1, wherein the motorized assembly comprises a varying speed motor to control a rate of retraction of the sheath.

5. The electrical drive system according to claim 3, wherein the pulley is configured to rotationally move to exert a tensile force on the cable.

6. The electrical drive system according to claim 1, wherein the motorized assembly further comprises a gear assembly, the gear assembly being coupled to the motor and a pulley assembly.

7. The electrical drive system according to claim 1, wherein the motorized assembly comprises a bore, the bore extending along a longitudinal axis of the motorized assembly.

8. A motorized delivery system for delivering and deploying an expandable endoluminal prosthesis, the system comprising:
an inner dilator having a proximal end and a distal end;
an elongate sheath having a proximal end, a distal end, and an inner lumen defining an inner surface, the distal end of the sheath being slidably disposed over the inner dilator;
an electrical drive mechanism comprising a motorized pulley assembly, the assembly being removably coupled and in mechanical communication with the sheath, whereby actuation of a motor causes the motorized pulley assembly to pull the elongate sheath proximally over the inner dilator.

9. The system according to claim 8, wherein the motorized pulley assembly further comprises one or more pulleys having one or more cables coupled to each of the one or more pulleys.

10. The system according to claim 9, wherein the motorized pulley assembly further comprises a gear assembly, the gear assembly configured to engage with the one or more pulleys.

11. The system according to claim 10, the motorized pulley assembly further comprising a worm gear engaging with a worm.

12. The system according to claim 9, wherein each of the one or more cables has a proximal end and a distal end, the distal end being coupled to the sheath and the proximal end being coupled to the pulley.

13. The system according to claim 11, wherein the worm gear is configured to engage with the one or more pulleys.

14. The system according to claim 8, wherein the electrical drive mechanism is configured to receive a guidewire.

15. The system according to claim 9, wherein at least a portion of each of the one or more cables is housed in a tubing.

16. The system according to claim 9, wherein the motorized pulley assembly further comprises one or more pulleys having one or more cables coupled to each of the one or more pulleys, further wherein the motorized pulley assembly further comprises a gear assembly, the gear assembly configured to engage with the one or more pulleys, further wherein the motorized pulley assembly comprises a worm gear engaging with a worm, further wherein each of the one or more cables has a proximal end and a distal end, the distal end being coupled to the sheath and the proximal end being coupled to the pulley, further wherein the worm gear is configured to engage with the one or more pulleys, further wherein the electrical drive mechanism is configured to receive a guidewire, further wherein at least a portion of each of the one or more cables is housed in a tubing, and further wherein the one or more pulleys drives the one or more cables to retract the sheath.

17. A method of deploying an expandable endoluminal prosthesis, the method comprising the steps of:
providing a prosthesis delivery system comprising an expandable prosthesis, an inner dilator and an elongate sheath, the prosthesis being disposed in a compressed configuration between the inner dilator and the elongate sheath;
providing an electrical drive system comprising a motorized assembly having a motor, gear assembly, and a pulley assembly, the electrical drive system being removably coupled to a proximal end of the delivery system;
actuating the motor,
driving the pulley assembly; and
retracting the sheath.

18. The method according to claim 17, wherein the driving the pulley assembly step comprises rotationally moving a pulley to exert a tensile force on a cable coupled to the pulley.

19. The method according to claim 17, further comprising the step of advancing a guidewire through a bore of the electrical drive system.

20. The method according to claim 17, wherein the retracting of the sheath is incrementally controlled.
